(19) United States
(12) Patent Application Publication
(10) Pub. No.: US 2022/0047273 A1
(43) Pub. Date: Feb. 17, 2022

(54) WINDLASS PEN

(71) Applicant: Recon Medical, LLC, Redding (CA)

(72) Inventors: Derek Parsons, Redding, CA (US); Zhang Yanfeng, Hengshui (CN)

(21) Appl. No.: 16/992,951

(22) Filed: Aug. 13, 2020

(51) Int. Cl. A61B 17/132; B43K 29/20

(52) U.S. Cl. CPC A61B 17/1327; B43K 7/00; B43K 29/20

(57) ABSTRACT

A tourniquet includes windlass having a cavity and a writing instrument configured to be stored in the cavity. The writing instrument is retained in the cavity through a friction fit and without threads, such that a frictional force decreases as the writing instrument is removed from the cavity.

### FIELD

This disclosure relates generally to tourniquets, and more particularly to windlasses of tourniquets.

### BACKGROUND

A tourniquet is a constricting band placed around a limb and tightened to eliminate arterial flow past the device. Simply tying a constricting band or material tightly around a limb rarely provides enough pressure to occlude arterial flow. To stop bleeding requires a mechanical advantage, and to stop bleeding, tightening the constricting band or material is necessary. A windlass or twist stick is conventionally used to tighten the constricting band or material.

Arterial blood flow (from the heart to extremities) into a limb is usually at a high-pressure and requires high pressure deep in the limb to occlude or stop the arterial flow. Venous flow (from the extremity to the heart) is usually at a low pressure from the limb and returning the blood back to the heart and requires less pressure than arterial flow to stop flow of the blood.

Material wrapped tightly around a limb, but without a “twist stick” or windlass to apply mechanical tension to the material, is a venous (low pressure) constricting band at best. Although arterial flow still occurs when material is merely wrapped tightly around a limb, which moves blood into the injured limb, the venous system is occluded or mostly occluded and can't return blood to the body. The blood that is flowing past the material wrapping through the artery will flow into the limb and will exit the limb through the wound in the limb, and thus out of body, resulting in blood loss. For wounded patients, that is detrimental if not fatal, but for taking blood samples that is desirable, where the phlebotomist places a “venous constricting band” around an arm to occlude the venous system which engorges the veins in the limb, making it easier to draw blood from enlarged veins. Thus, tourniquets without windlasses are venous constricting bands because windlasses are necessary to prevent hemorrhaging through the wound.

Tourniquets should be removed as soon as safely possible. Having a tourniquet applied for more than 6 hours is associated with distal tissue loss. According to at least one authority a tourniquet that has been in place for more than six hours should be left in place until the patient arrives at a medical facility. The personnel at the medical facility need to know the amount of time that the tourniquet has been in place in determining their treatment. Thus, it is critical to record the time that a tourniquet is applied. Many tourniquets have a place designated to record the time applied. However, because tourniquets are typically applied in the field outside of medical facilities, often in combat, disasters, or other emergency situations, a writing instrument capable of writing on the tourniquets may not be readily available.

U.S. Pat. Pub 20190216471 by Strattner for a “Tourniquet Windlass Device” describes a pen that can be used as a tourniquet windlass. The pen cannot be used to record on the tourniquet the time that the tourniquet was applied, because the pen is the windlass. The device of U.S. Pat. Pub 20190216471 is not directed to solving the problem of writing the time when the tourniquet is applied. The device combines the functionality of a pen and a windlass to “render the device desirable for persons who need to balance the weight and cumbersomeness of carrying multiple devices with the need to have a minimum number of functionalities within the set of devices that they carry.”

U.S. Pat. Pub. No. 20180168663 of Hill for a “Windlass Tourniquet With Embedded Writing Implement” describes a writing instrument that screws into a windlass. The writing instrument has male threads that mate with female screws in a tourniquet windlass. Alternatively, the writing instrument may be snapped into place in the windlass.

U.S. Pat. Pub. No. 20160367262 to Burke et al. for a “Tourniquet and Windlass Assembly and Method” describes a marking device, a grease pencil, having a cap on one end, which cap screws into a windlass. The cap has male threads that mate with female screws in a tourniquet windlass.

### BRIEF DESCRIPTION

The above-mentioned shortcomings, disadvantages and problems are addressed herein, which will be understood by reading and studying the following specification.

In one aspect, a tourniquet to restrict a flow of blood in a body part includes a windlass having a cavity into which a writing instrument can be inserted. The writing instrument can be used, for example, for recording the time that the tourniquet was applied. The writing instrument is maintained within the cavity by a non-localized friction fit rather than being threaded into the cavity or held by a detent that requires a large initial force to overcome. The frictional force is determined by the design and is not dependent on how the writing instrument was last inserted.

Apparatus, systems, and methods of varying scope are described herein. In addition to the aspects and advantages described in this summary, further aspects and advantages will become apparent by reference to the drawings and by reading the detailed description that follows.

### BRIEF DESCRIPTION OF THE DRAWINGS

FIG. 1 is an isometric diagram of a tourniquet with a windlass that stores a writing instrument;

FIG. 2 is a diagram of a side view of the tourniquet of FIG. 1;

FIG. 3 is a diagram of a top view of the tourniquet of FIG. 1 with the restraining strap unsecured;

FIG. 4 is a diagram of a top view of the tourniquet of FIG. 1 with the restraining strap secured;

FIG. 5 is an end view of a windlass without a writing instrument inserted;

FIG. 6 is a side view of a windlass showing how a writing instrument is inserted;

FIG. 7 is a block diagram of an embodiment of a writing instrument for inserting into a windlass; FIG. 7A is a detail view showing a portion of FIG. 7 enlarged;

FIG. 8 is a block diagram of another embodiment of a writing instrument for inserting into a windlass;

FIG. 9 is a block diagram of another embodiment of a writing instrument for inserting into a windlass;

FIG. 10 is a block diagram of another embodiment of a writing instrument for inserting into a windlass;

FIG. 11 shows the friction versus position of a prior art snap holding in the writing instrument;

FIG. 12 shows qualitatively friction versus position of some embodiments of writing instruments;

FIG. 13 shows qualitatively friction versus position of some other embodiments of writing instruments; and

FIG. 14 shows an enlarged view of the writing end of a writing instrument tip;

FIG. 15 is a flowchart of a method to use a tourniquet having a writing instrument stored in the windlass.

### DETAILED DESCRIPTION

In the following detailed description, reference is made to the accompanying drawings that form a part hereof, and in which is shown by way of illustration specific implementations which may be practiced. These implementations are described in sufficient detail to enable those skilled in the art to practice the implementations, and it is to be understood that other implementations may be utilized and that logical, mechanical, electrical and other changes may be made without departing from the scope of the implementations. The following detailed description is, therefore, not to be taken in a limiting sense.

The detailed description is divided into three sections. In the first section, problems with the prior art are described. In the second section, apparatus of implementations are described. In the third section, implementations of methods are described. Finally, in the fourth section, a conclusion of the detailed description is provided.

### Problems with the Prior Art

The systems of Hill and Burke et al. retain the writing instrument in the windlass using threads. That is, the pen screws into the windlass. Unscrewing the tourniquet pens of Hill and Burke from the windlass requires manual dexterity and multiple application of fine motor skills, which can be difficult to achieve in combat or other stressful situations. It is known that stress reduces dexterity and fine motor skills. See, for example, T. Cuper “Modeling the Effects of Stress: An Approach to Training,” https://ntrs.nasa.gov/archive/nasa/casi.ntrs.nasa.gov/20100012855.pdf, which states: “In the case of a combat medic, stress levels can be expected to be extremely high as the lives of both the medic and his comrades are at risk. In addition, the medic's performance requirements are complex, demanding both gross (e.g. running) and fine (e.g. applying a tourniquet, firing a weapon) motor skills as well as unaffected cognitive functioning. It is known that under stress, fine motor skills and dexterity can be greatly reduced.”

Moreover, the initial force to begin unscrewing the marking device varies with the torque applied when the marking device was last inserted. Tourniquets are typically applied to patients that are losing a great deal of blood, and the hands of the field medic or first aider are often slippery with blood. This can make it difficult to apply sufficient torque to unscrew the writing device from the windlass, particularly if the writing device was over torqued when it was last inserted.

A snap, such as described by Hill, requires a greater initial force to overcome the snap, which requires a tight user grip that may not be possible with the user's hand wet with blood and fine motor skills compromised by stress. Moreover, if the pen is inadvertently snagged by something in the environment, such as dense vegetation, the pen can move sufficiently far to overcome the snap. Once the localized snap force is overcome, there is nothing to prevent the pen from falling out of the windlass.

Tourniquets are typically used in the field, in conditions in which they are exposed to dirt, mud, and other contaminants. Such contaminates can clog threads and snaps, making it more difficult to remove the pen, especially under stressful conditions.

### Apparatus Implementations

In this section, the particular apparatus of such an implementation are described by reference to a series of diagrams.

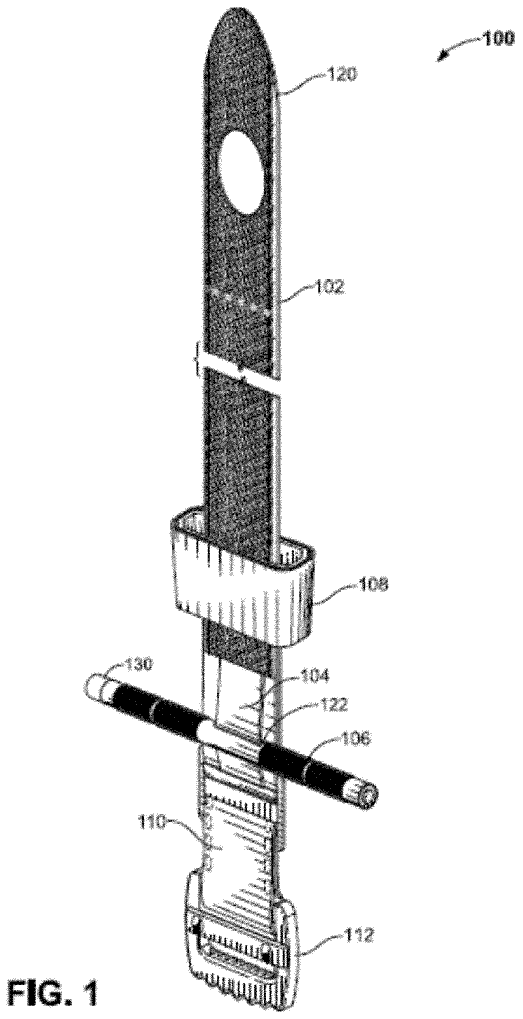

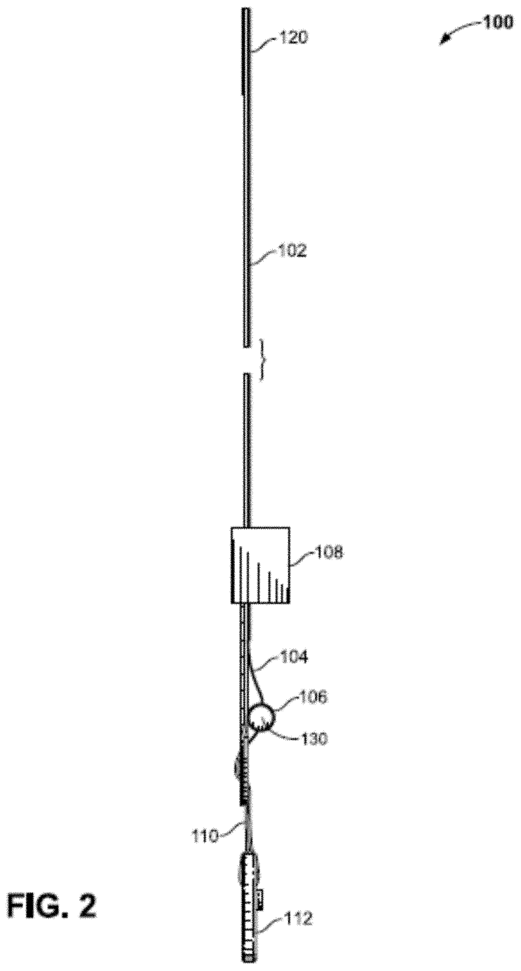

FIG. 1 is an isometric diagram of a tourniquet 100. FIG. 2 is a right side elevation of the tourniquet 100. In FIGS. 1 and 2, the tourniquet 100 includes a first elongated member or a belt 102, a second elongated member (inner tightening member or inner strap) 104, a windlass 106 and a securing mechanism 108. The tourniquet 100 can be applied to a limb, as for example, a leg or arm, and then tightened to restrict the flow of blood to the limb. The tourniquet 100 also includes a connector 110 and a buckle 112.

To apply the tourniquet 100 to the limb, the user simply wraps the tourniquet around the subject appendage, loops the first end 120 of the belt 102 through the buckle 112, pulls the tourniquet 100 reasonably tight. FIGS. 1 and 2 depict the tourniquet 100 when the first end 120 has not been looped through the buckle 112. The buckle 112 provides the advantage of the tourniquet being quickly adjustable when in use to accommodate a variety of size appendages, as for example, from a person's thigh to a person's forearm.

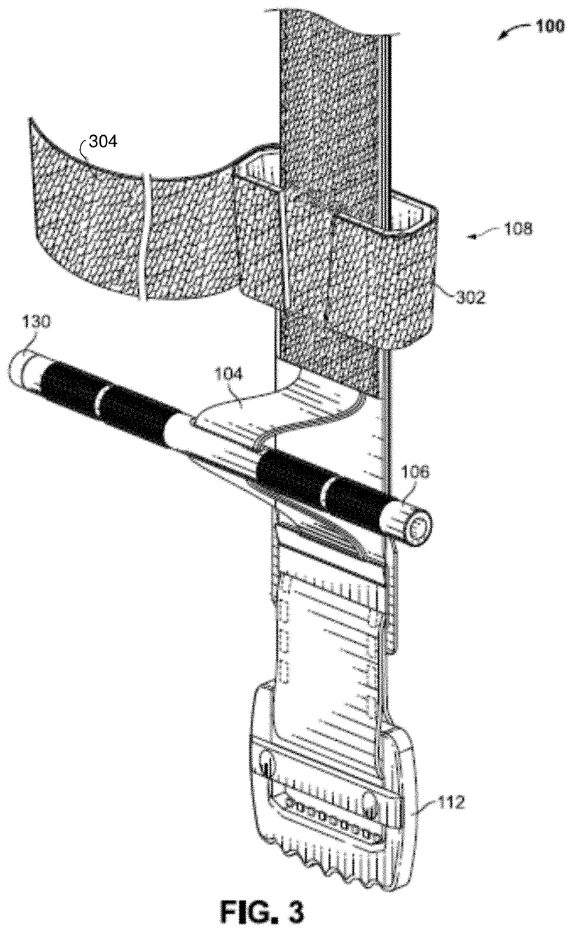

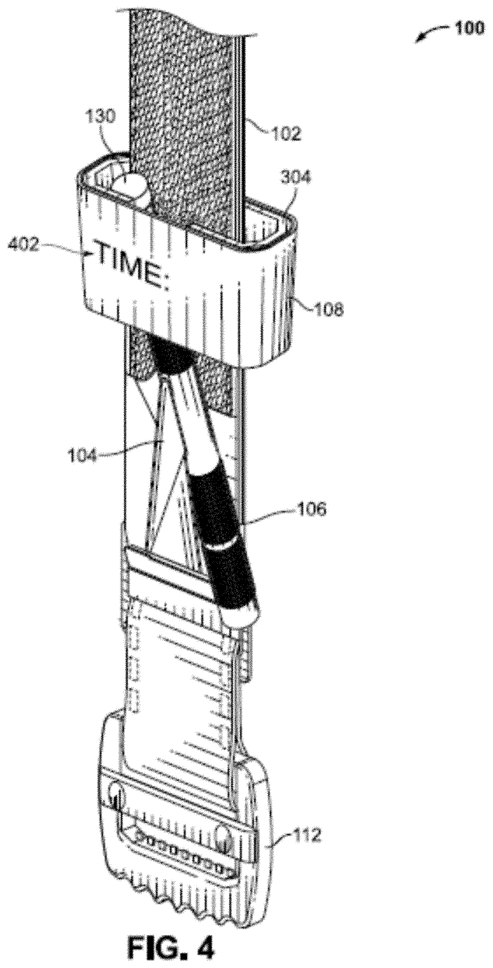

A windlass 106 includes a slot 122 through passes to the inner strap 104 as seen in FIG. 2. The windlass 106 can be used to tighten the belt 102, such as by winding the windlass 106 to develop a tension force in the inner strap 104, which then tightens belt 102. After the windlass 106 has been sufficiently tightened to restrict the arterial blood flow in the limb, the windlass 106 may be secured by the securing mechanism 108 in FIG. 3. The securing mechanism 108 comprises a windlass clip 302 covered with one portion of a hook-and-loop fastener. The windlass 106 after being rotated is captured within the windlass clip 302 to prevent the inner strap 104 from unwinding. A securing strap 304, including the mating portions of the hook-and-loop fasteners on the windlass clip 302, attaches to the windlass clip as shown in FIG. 4 to maintain the windlass 106 within the windless clip, thereby maintaining the tension in the inner strap 104. Windlass 106 includes a writing instrument 130 that will be described in more detail below.

As shown in FIG. 4, a markable surface suitable for being written upon by writing instrument 130, is provided, such as on securing strap 304. A marking 402, such as the words “TIME:” on securing strap 304 can be used to indicate where to record the time the tourniquet was applied to the patient. Securing strap 304 preferably has a surface compatible with the marking substance used in writing instrument 130, that is, writing instrument can permanently write on securing strap. The markable surface provides a medium for the user of the tourniquet to quickly, conveniently and easily record information during the use of the tourniquet 100, such as the time of the day that the tourniquet 100 was applied to a patient. The time of the day that the tourniquet 100 was applied to a patient is important to people who attend to and provide healthcare to the patient because tourniquets can only be applied to a patient to a limited amount of time without causing tissue damage to the patient. The ability to record the time of the day that the tourniquet 100 was applied to a patient can have a significant impact in the quality of the healthcare received by the patient and can significantly impact the medical recovery of the patient. In other implementations, the surface that is suitable for writing by a pen may be located on other portions of the tourniquet 100, such as the belt 102.

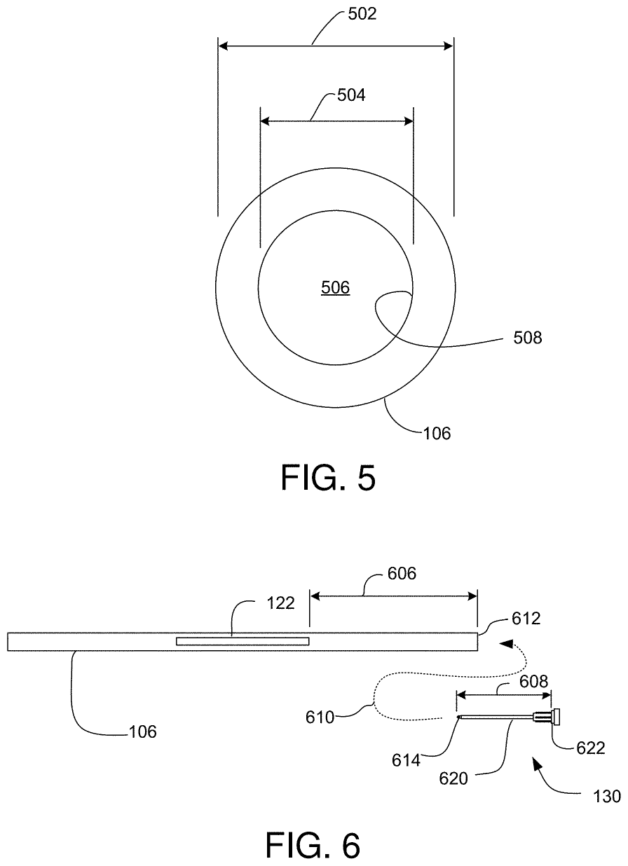

FIG. 5 shows an end-in view of the windlass 106, which is in the form of a hollow cylinder having an outer diameter 502, preferably about 0.340″ and an inner diameter 504, preferably about 0.259″. The hollow cylinder defines a cavity 506 having a cylindrical interior wall 508. Interior wall 508 can be smooth or can be textured to increase friction, but is preferably not threaded.

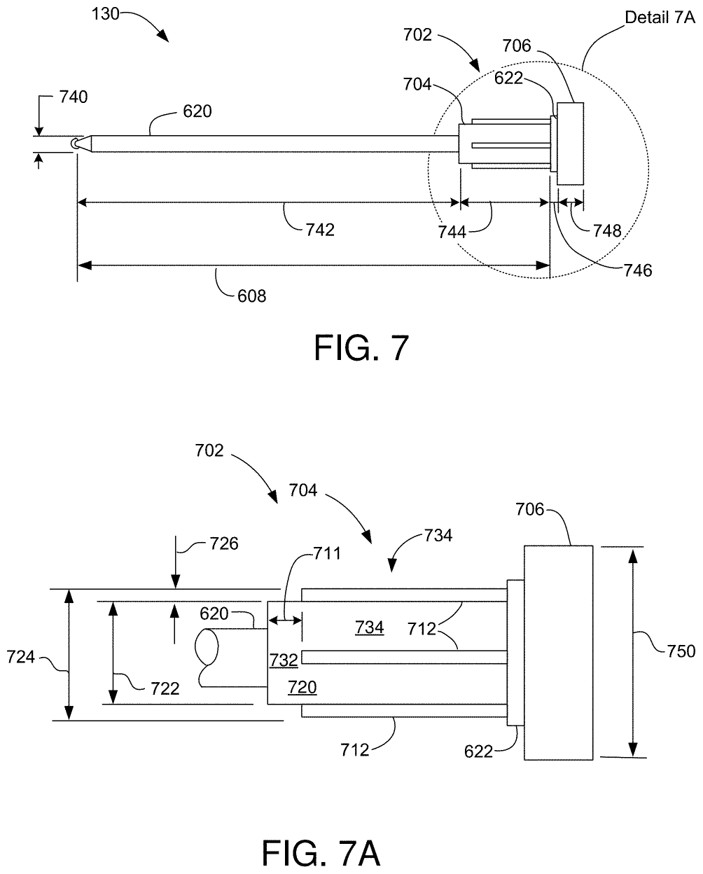

FIG. 6 shows the windlass 106 including slit 122 through which the inner strap 104 passes. The slit 122 begins at a distance 606 from the end 612 of windlass 106. FIG. 6 also shows writing instrument 130 which has a length 608 from the end 614 of shaft 620 to a standoff 622. Standoff 622 and length 608 are shown enlarged in FIG. 7. Writing instrument 130 will therefore only penetrate into cavity 506 a length equal to the distance 608. Parts of the writing instrument 130 are shown more clearly in FIGS. 7-10. Arrow 610 indicates that the writing instrument 130 is inserted into windlass cavity 506 from the end of windlass 106. Length 608 is less than distance 606 so that when writing instrument 130 is inserted into cavity 506, the writing instrument 130 will not interfere with inner strap 104 (not shown) running through slit 122.

FIG. 7 is a diagram of an example writing instrument 130. The writing instrument 130 includes shaft 620, which is preferably composed of a material, such as copper, that will not rust or corrode. Shaft 620 has a diameter 740 that is smaller than the inner diameter 504 of cavity 506. For example, a preferred diameter 740 is between 0.05″ and 0.2″, for example 0.088″. The shaft is preferably between about 1″ and about 2″ long, preferably about 1.630″ long. The shaft 620 is attached to a head 702, which is shown in more detail in Detail 7A. Head 702 is preferably made of a polymer material, such as a plastic or rubber compound, that is hard yet slightly compressible. Head 702 includes a friction fit portion 704, stand off portion 622, and an end portion 706. Friction fit portion 704 provides a friction fit with the interior walls 508 of cavity 506. Friction fit portion 704 has an overall diameter 724 preferably between about 0.1″ and about 0.3″, preferably about 0.191″. Friction fit portion 704 has a length 744 preferably between about 0.2″ and about 0.6″, preferably about 0.454″. Standout portion 622 has a length 746 preferably between about 0.02″ and about 0.06″, preferably about 0.033″. Standout portion 622 has a diameter preferably between about 0.2″ and about 0.4″, preferably about 0.259″. End portion 706 has a length 748 preferably between about 0.07″ and about 0.3″, preferably about 0.115″, and a diameter preferably between about 0.25″ and about 0.5″, preferably about 0.340″.

FIG. 7A shows that friction fit portion 704 includes a cylindrical-shaped smooth portion 720 having a diameter 722 preferably between about 0.15″ and 0.4″, preferably about 0.260 in. Friction fit portion 704 comprises a contact portion 734 that includes ridges 712 that extend radially a distance 726 from the smooth portion 720. Ridges run from standoff portion 622 but preferably stop a distance 711 before reaching the end of friction fit portion 704, thereby providing gaps 732 that facilitate inserting the friction fit portion into the windlass cavity. An overall diameter 724 is defined as the diameter of a cylinder that encloses the ridges 712 or other features that extend above smooth portion 720. The overall diameter 724 is preferably slightly larger than the inner diameter of 504 of cavity 506 to provide an interference fit with the cavity. By making the overall diameter 724 slightly greater than the inner diameter 504 of cavity 506, the ridges are compressed when the friction fit portion 704 is pushed into cavity 506 to produce friction between friction fit 704 and the interior wall of cavity 506 to secure the writing instrument 130 within windlass 106 when writing instrument 130 is not in use.

In one implementation, ridges 712 are parallel to the axis of shaft 620 and evenly spaced circumferentially about smooth cylindrical portion 720 at 60 degrees from each other. Each of ridges 712 extends preferably between about 0.005″ and about 0.10″, above the smooth portion 710. Other configurations can be used, such as differently-shaped ridges or no ridges at all, in which the friction is provided between smooth cylindrical portion 720 and interior wall 508.

The frictional force required to remove writing instrument from cavity 506 depends on several factors, including the materials of which the ridges 712 and the interior wall 508 of cavity 506 are composed, the surface textures of the ridges and the interior wall of cavity 506, the contact area between the ridges and the inner surface of the cavity, and height of ridges 712 which defines the interference fit and determines the compression of the ridges. Additional ridges would increase the contact surface area, which would increase the friction. Skilled person can adjust the friction factors to produce a friction fit that is sufficient to secure the writing instrument 130 in windlass 122, but that permits removal of the writing instrument by a user having reduced dexterity due to stress and/or having hands wet and slippery with blood.

Ridges 712 preferably do not extend to the end of friction fit portion 704 from which the shaft 620 extends, but as described above provide for the gap 732 having a length 711 of preferably between about 0.01″ and about 0.10″, between the end of friction fit portion 704 from which shaft 620 extends and the beginning of ridges 712. The gap facilitates the insertion of the friction fit portion 704 into the cavity 506 of the windlass 106.

As the writing instrument is withdrawn, once the static friction is overcome, the friction decreases smoothly as the area of the ridges having contact with the cavity is reduced. The smooth reduction in friction makes it easier for a user having hands that are wet and slippery with blood to withdraw the writing instrument to write information on the retaining strap. Designing the friction fit portion 720 so that the friction reduces smooths eliminates the requirement for a large initial force, such as the force that would be required to overcome the resistance of a snap or detent.

With a snap or detent, there is a large initial force to overcome and then essentially no frictional force as the writing instrument is further withdrawn. In the embodiment of FIG. 7, the is a smooth decrease in force over the length as the writing instrument is withdrawn. The force continues is present but continues to decrease until the ridges no longer contact the inside of the windlass.

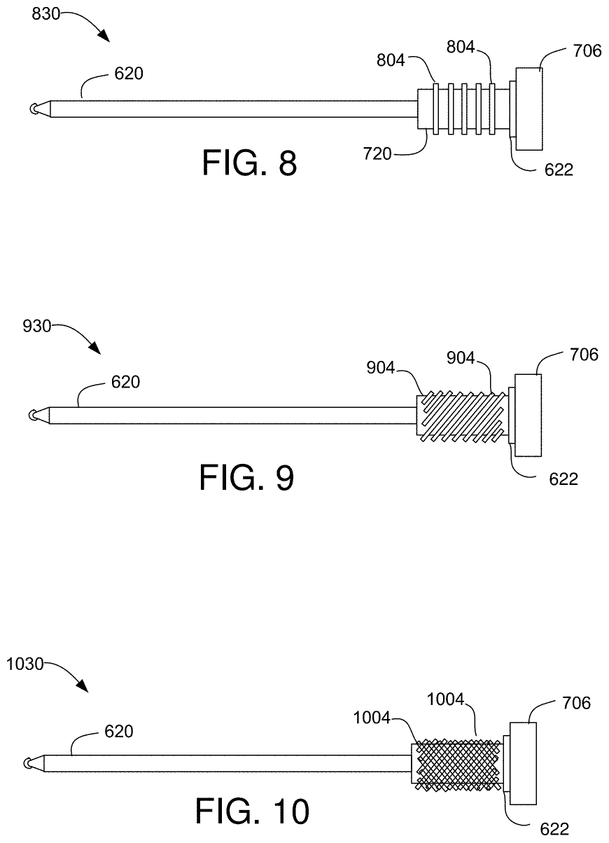

FIG. 8 shows an alternative embodiment of a writing instrument 830. Ridges 804 are equally spaced, each in a plane perpendicular to the axis of shaft 620. As writing instrument 130 is withdrawn, subsequent ones of ridges 804 lose contact with the interior wall of cavity 506. The friction reduces incrementally as each one of ridges leaves cavity 506. The contribution to the overall friction from each of ridges 804 is relatively small, so although the friction reduces incrementally, each incremental change in friction is relatively small and so to the user, it feels like the friction is being reduced smoothly.

FIG. 9 shows an embodiment of a writing instrument 930 in which each of ridges 904 is in a plane angled with respect to the axis of shaft 620. FIG. 10 shows another embodiment of a writing instrument 1030 in which ridges 1004 comprise a first set of ridges, each in a plane tilted at a first angle with respect to the axis of shaft 620 and a second set of ridges, each in a plane tilted at a second angle with respect to the axis of shaft 620, the first and second sets of ridges overlapping. Other patterns of ridges or other protrusions or textures, such as raised dots, diamonds, or squares, can also be used.

Ridges 704, 804, 904, and 1004, referred to below ridges X04, preferably extend between about 0.01″ to 0.1″, preferably about 0.06″, above the major cylindrical surface of friction fit portion 710. When inserting the writing instrument into the interior cavity, the ridges are compressed slightly to maintain the writing instrument 130 within the interior cavity 506. The friction that keeps the writing instrument within the windlass cavity is determined by the materials of which the windlass and the ridges X04, as well as the number and geometry of the ridges. As the writing instrument is withdraw, the friction decreases in a relatively smooth manner as the contact area between the ridges and the interior wall of cavity 506 is reduced. The incremental reduction in friction makes it easier for a user having hands that are wet with blood to withdraw the writing instrument to write information on the retaining strap. Because the friction is spread amount the ridges, there is no initial large force needed to overcome.

In some implementations the writing instrument includes a ball point pen. The pen uses gel-type, waterproof, permanent ink or other marking substance. In some implementations, the windlass pen comprises a felt tip pen, a grease pencil, or a carbon pencil. The shaft is preferably made of copper or other material that will not rust or corrode. The writing substance may be stored within shaft 620.

The diameter of the standoff portion 622 is slightly larger than the inner diameter of the windlass cavity so that the stand-off portion will not go into the cavity 506, thereby preventing the writing instrument 130 from being inserted into the cavity 506 in windlass 106 up to the end 706. Keeping the end 706 positioned slightly way from the end of the windlass 106 facilitates grasping of the knurled end portion by a user.

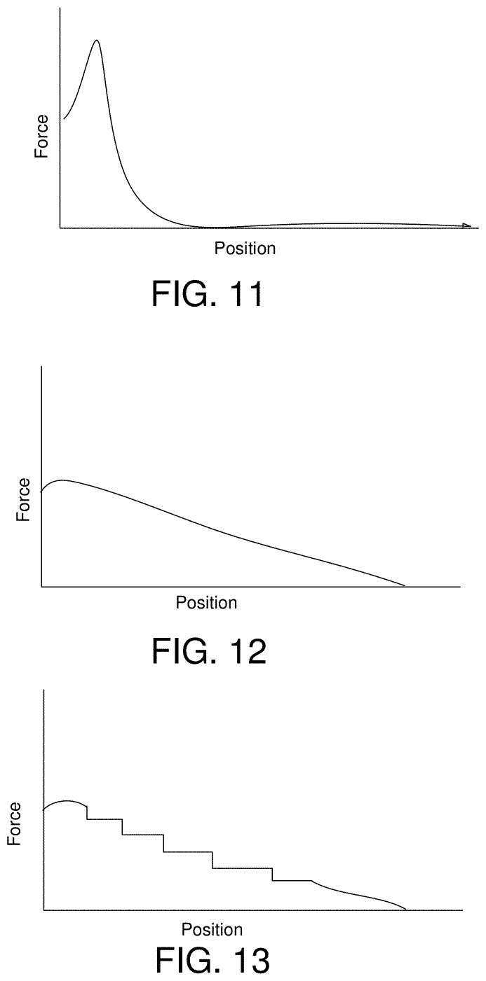

FIGS. 11, 12, and 13 show qualitatively the frictional force required to move the writing instrument versus the position of the writing instrument. FIG. 11 represents the force from a prior art snap or detent mechanism holding in the writing instrument. As shown in FIG. 11, a large initial force is required to overcome the snap force, and then there is little or no friction to remove the writing instrument after the initial snap friction is overcome. Because in a prior art snap or detent the friction is concentrated at one point, the pre-set friction force must be relatively high to prevent the writing instrument from falling from the windlass in the case of an inadvertent force being applied over the short distance. In embodiments in which the force is required to be applied over a distance, the frictional force can be lower because the writing instrument will not fall out of the windlass if it is inadvertently displaced slightly.

FIG. 12 shows the friction versus position of embodiments such as FIG. 7, FIG. 9, or FIG. 10. The friction force decreases smoothly as the writing instrument is withdrawn because there is less contact with the interior cavity of the windlass. FIG. 13 shows how the friction decreases gradually but incrementally in an embodiment such as FIG. 8, in which the friction decreases in an increment as a ring loses contact with the interior contact of the windlass cavity. The friction force is non-localized, that is, the contact between the writing instrument and the interior cavity of the windlass is distributed along the length of the cavity and so the frictional force is distributed and reduces as the writing instrument is withdrawn and the contact area is reduced. In some embodiments, friction resists the movement of the writing instrument for at least 25%, at least 50%, or at least 75% of the travel distance of the friction fit portion as the writing instrument moves as it is withdrawn. In some embodiments, friction reduces gradually for at least 25%, at least 50%, or at least 75% of the travel distance of the friction fit portion as the writing instrument moves as it is withdrawn. In some embodiments, the friction is initially distributed over a contact area having a length, measured in the along the axis of shaft 610, of at least 3 mm, at least 5 mm or at least 7 mm.

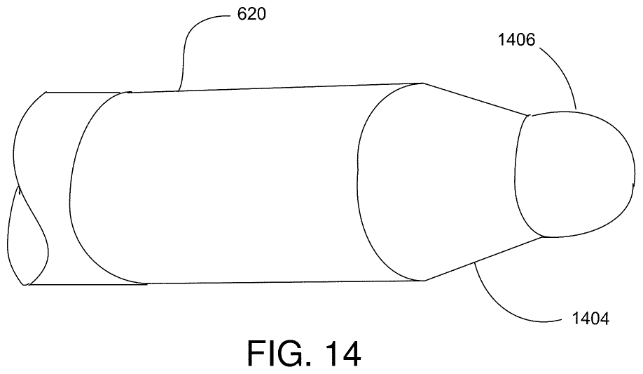

FIG. 14 shows an enlarged view of a portion of shaft 620 of an embodiment in which the writing instrument comprises a ball point pen. FIG. 14 shows that shaft 620 is cylindrical along most its length and includes a tapered portion 1404 toward the end from which the ink or other marking substance leaves the writing instrument 130. Because a tourniquet may be carried in first aid keep for an extended period of time before it is used, the ink or other marking substance should not dry out during storage. A protective substance 1406, such as a blob of wax or other impermeable material keeps the ink or other marking substance from drying out during storage and yet is readily removed when the user begins to mark with the writing instrument 130.

### Method Implementations

The previous section describes an example of a tourniquet having a writing instrument configured to be stored in a windlass. In this section, the particular methods of such an implementation are described by reference to a series of flowcharts.

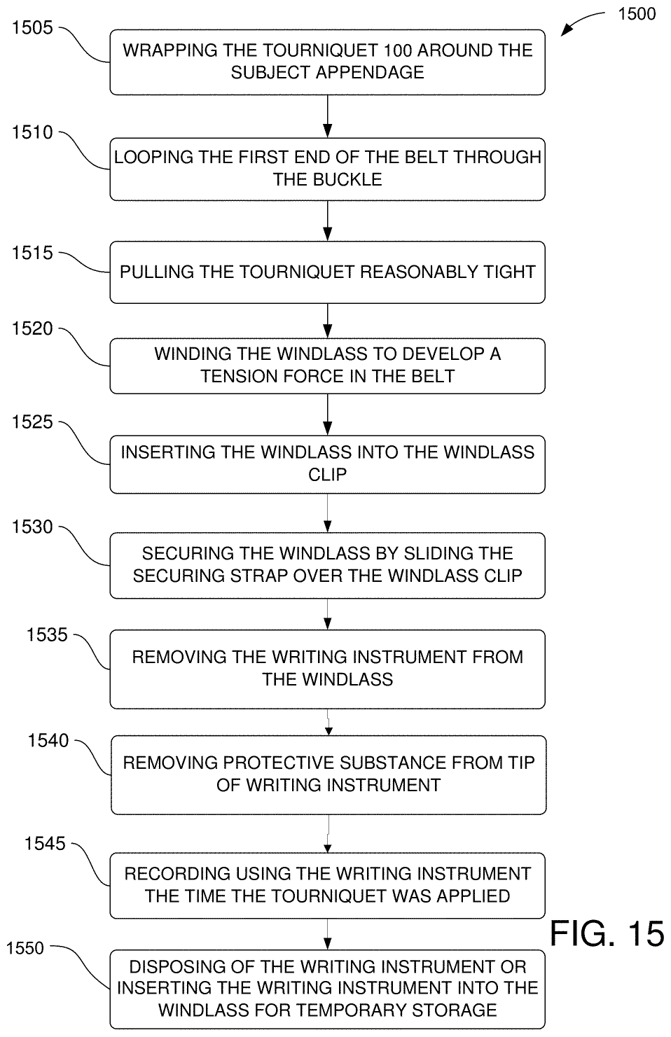

FIG. 15 is a flowchart of an example method 1500 to use the tourniquet in FIGS. 1-11 to restrict arterial blood flow in a limb.

Method 1500 includes in step 1505 wrapping the tourniquet 100 around the subject appendage and then step 1510 includes looping the first end 120 of the belt 102 through the buckle 112. The tourniquet 100 is pulled reasonably tight in step 1515.

Step 1520 includes winding the windlass 106 to develop a tension force to tighten inner strap 104 which tightens the belt 102. After the windlass 106 has been sufficiently tightened to restrict the arterial blood flow in the limb, the windlass 106 is inserted into windlass clip 302 in step 1525 to prevent inner belt 104 from unwinding. In step 1530, the opening in the windlass clip is covered with the retaining strap 108, which includes hook-and-loop fasteners that mate with the hook-and-loop fasteners on the windlass clip to prevent the windlass from coming out of the windlass clip.

In step 1535, writing instrument 130 is removed from windlass 106 and in step 1540, the protective substance is removed from the tip of the writing instrument. The protective substance can be removed by merely starting to write with the writing instrument, with the protective substance coming off as the writing instrument contacts and moves relative to the markable surface. In step 1545, a user records the time at which the tourniquet was applied and any additional notes. The time can be recorded on the tourniquet itself, such as on the restraining strap 304. Alternatively, the time can be recorded elsewhere, such as on the clothing or skin of the patient. In step 1550, writing instrument 130 is then discarded or temporarily reinserted into windlass 106. If writing instrument 130 is temporarily reinserted in windlass cavity 506, it should eventually be disposed of because with the protective substance removed, the ink or other marking substance may eventually dry and render the writing instrument unusable.

Although the description described a tourniquet with an inner and outer strap, the writing instrument can be used with a windlass of any type of tourniquet.

### CONCLUSION

A tourniquet having a windlass that stores a writing instrument is described. Although specific implementations are illustrated and described herein, it will be appreciated by those of ordinary skill in the art that any arrangement which is calculated to achieve the same purpose may be substituted for the specific implementations shown. This application is intended to cover any adaptations or variations. For example, although described in medical terms, one of ordinary skill in the art will appreciate that implementations can be used in industrial environments or any other environment that provides the required function.

In particular, one of skill in the art will readily appreciate that the names of the methods and apparatus are not intended to limit implementations. Furthermore, additional methods and apparatus can be added to the components, functions can be rearranged among the components, and new components to correspond to future enhancements and physical devices used in implementations can be introduced without departing from the scope of implementations. One of skill in the art will readily recognize that implementations are applicable to future tourniquet devices, different pens, and new buckles.

The terminology used in this application is meant to include all tourniquets, buckles, belts and connecting members and alternate technologies which provide the same functionality as described herein

1. A tourniquet to restrict a flow of blood in a limb, the tourniquet comprising:
a strap for wrapping around a portion of a limb;
a windlass for tightening the strap around the portion of the limb to suppress blood flow to and from that limb, the windlass having a cavity having an inner diameter and an interior wall; and
a writing instrument adapted to be secured in part in the windlass cavity without threads, the writing instrument including:
a shaft for containing a marking substance;
a head, attached to the shaft, the head comprising
a friction fit portion, the friction fit portion providing a non-localized friction fit with the inner wall of the windlass cavity;
a standoff portion; and
an end portion, separated from the end of the windlass by the standoff portion when the writing instrument is inserted into the windlass cavity, the end portion including a knurled surface for facilitating grip by a user to overcome the friction and remove the writing instrument from the windlass.

2. The tourniquet of claim 1, wherein friction fit portion includes a contact portion, the contact portion comprising ridges forming an interference fit with the cavity walls.

3. The tourniquet of claim 2, wherein the ridges are parallel to the long axis of the shaft.

4. The tourniquet of claim 1, wherein the friction fit portion includes a contact portion adapted to provide continuous friction as the contact portion is withdrawn from the windlass cavity.

5. The tourniquet of claim 1, wherein the friction fit portion includes a contact portion adapted to continue to provide friction as the writing instrument is withdrawn a distance of at least 3 mm from the windlass cavity.

6. The tourniquet of claim 1, wherein the friction fit portion includes a contact portion adapted to continue to provide friction as the writing instrument is withdrawn a distance of at least 6 mm from the windlass cavity.

7. The tourniquet of claim 2, wherein the ridges are concentric with the long axis of the shaft, the ridges forming an interference fit with the cavity walls.

8. The tourniquet of claim 1, wherein the friction fit portion includes a contact portion configured such that the friction between the contact portion and the interior wall of the windlass cavity decreases smoothly as the contact portion is pulled out from the windlass.

9. The tourniquet of claim 1, wherein the friction fit portion includes a contact portion configured such that the friction between the contact portion and the interior walls of the cavity of the windlass decreases in multiple incremental steps as the contact portion is pulled out from the windlass.

10. The tourniquet of claim 1, further comprising a restraining mechanism adapted for restraining the windlass after it has been tightened, the restraining mechanism including a clip and a restraining strap, the restraining strap having a surface that can be written upon by the writing instrument.

11. The tourniquet of claim 1, wherein the wherein the shaft contains a permanent and/or waterproof ink or other marking substance.

12. The tourniquet of claim 1, wherein the shaft contains a gel-type ink.

13. The tourniquet of claim 1, wherein:
the belt includes an inner strap and an outer strap; and
the windlass includes a slot through which the inner strap passes, the inner strap tightening as the windlass is wound.

14. A pen for inserting into a tourniquet windlass, comprising:
a shaft for containing a marking substance;
a head, attached to the shaft, the head comprising a friction fit portion, the friction fit portion providing a non-localized friction fit with the inner wall of the windlass cavity;
a standoff portion; and
an end portion, separated when the writing instrument is inserted into the windlass cavity from the end of the windlass by the standoff portion when the writing instrument is inserted into the windlass, the end portion including a knurled edge for facilitating grip by a user to overcome the friction and remove the writing instrument from the withdraw.

15. The pen of claim 14, wherein the pen comprises a gel-type ink.

16. The pen of claim 14, wherein the friction fit portion comprises ridges.

17. The pen of claim 16, wherein the shaft has a longitudinal axis and the ridges are parallel to the longitudinal axis.

18. A method of employing a tourniquet, comprising:
providing a tourniquet having a writing implement
wrapping the tourniquet around a limb;
tightening the tourniquet using a windlass;
securing the windlass to prevent it from unwinding;
withdrawing a writing instrument from the end of the windlass by applying a force that decreases over a distance of at least 3 mm as the writing instrument is withdrawn from the windlass;
writing the time that the tourniquet was applied to the limb on a portion of the tourniquet or elsewhere, in which withdrawing the writing instrument from the end of the windlass does not include unscrewing the writing instrument from threads in the windlass.

19. The method of claim 18 in which withdrawing a writing instrument from the end of the windlass by applying a force that decreases over a distance of at least 3 mm as the writing instrument is withdrawn from the windlass comprises withdrawing a writing instrument by applying a force that decreases smoothly after overcoming a static frictional force as the writing instrument is withdrawn.

20. The method of claim 18 in which withdrawing a writing instrument comprises applying a force that decreases over a distance of at least 5 mm as the writing instrument is withdrawn from the windlass.
